(19) United States
(12) Patent Application Publication
(10) Pub. No.: US 2026/0154977 A1
(43) Pub. Date: Jun. 4, 2026

(54) MEDICAL IMAGING DATA PROCESSING APPARATUS AND METHOD

(71) Applicant: CANON MEDICAL SYSTEMS CORPORATION, Otawara-shi (JP)

(72) Inventor: Magnus WAHRENBERG, Edinburgh (GB)

(73) Assignee: CANON MEDICAL SYSTEMS CORPORATION, Otawara-shi (JP)

(21) Appl. No.: 18/966,204

(22) Filed: Dec. 3, 2024

(51) Int. Cl. G06V 20/70; G06V 10/25; G06V 10/34; G06V 10/77; G06V 10/94; G16H 30/40

(52) U.S. Cl. CPC G06V 20/70; G06V 10/25; G06V 10/34; G06V 10/7715; G06V 10/945; G16H 30/40; G06V 2201/03

(57) ABSTRACT

A medical image data processing method comprises:
displaying a map that represents a plurality of image rendering parameters or other image generation conditions;
setting an indicator to select one or more of the image rendering parameters or other image generation conditions;
inputting medical image data and the selected one or more of image rendering parameters or other image generation conditions into a model; and
outputting a rendered medical image or other medical image data generated using the selected one or more of image rendering parameters or other image generation conditions.

### FIELD

Embodiments described herein relate generally to a method and apparatus for processing medical imaging data.

### BACKGROUND

Volume rendering is used in many clinical applications. Typically, in volume rendering applications, most rendering parameters are set either using view interactivity, input boxes or sliders.

Image captioning networks, comprising multi-modal models (MMMs) are known as a suitable tool for extracting semantic information from images, including rendered images.

### BRIEF DESCRIPTION OF THE DRAWINGS

Embodiments are now described, by way of non-limiting example, and are illustrated in the following figures, in which:

FIG. 1 is a schematic illustration of an apparatus in accordance with an embodiment;

FIG. 2 is a flowchart illustrating in overview a method according to an embodiment;

FIG. 3 is a schematic illustration of a method of processing medical image data in accordance with an embodiment;

FIG. 4 is an illustration of a grid of rendered images in accordance with an embodiment;

FIG. 5 illustrates a parameter space in accordance with an embodiment;

FIG. 6 illustrates three representations of a parameter spaces in accordance with an embodiment;

FIG. 7 is an image of a user interface in accordance with an embodiment;

FIGS. 8a to 8d illustrates parameter spaces in accordance with an embodiment; and

FIG. 9 is a schematic illustration of a method in accordance with an embodiment.

### DETAILED DESCRIPTION

According to certain embodiments there is provided a medical image data processing method comprising:
displaying a map that represents a plurality of image rendering parameters or other image generation conditions;
setting an indicator to select one or more of the image rendering parameters or other image generation conditions;
inputting medical image data and the selected one or more of image rendering parameters or other image generation conditions into a model; and
outputting a rendered medical image or other medical image data generated using the selected one or more of image rendering parameters or other image generation conditions.

According to certain embodiments there is provided a medical image data processing apparatus comprising processing circuitry configured to:
display a map that represents a plurality of image rendering parameters or other image generation conditions;
set an indicator to select one or more of the image rendering parameters or other image generation conditions;
input medical image data and the selected one or more of image rendering parameters or other image generation conditions into a model; and
output a rendered medical image or other medical image data generated using the selected one or more of image rendering parameters or other image generation conditions.

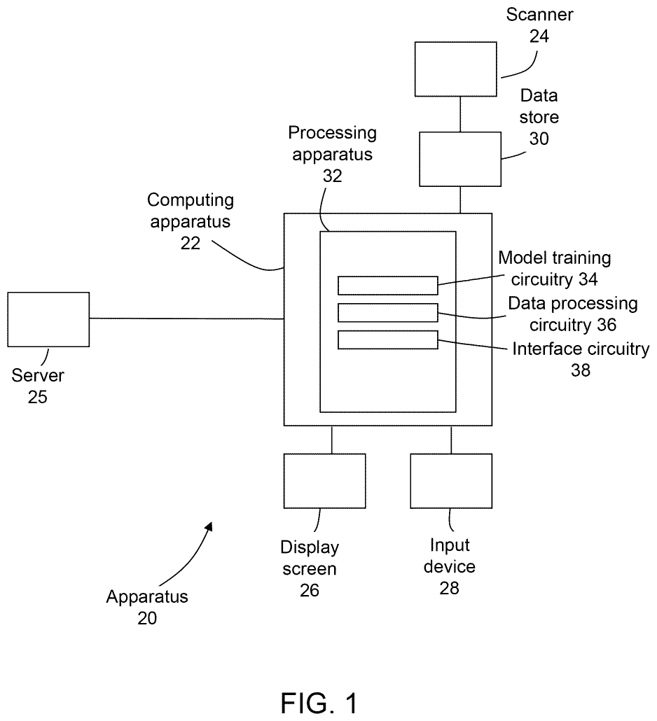

A medical imaging data processing apparatus 20 according to an embodiment is illustrated schematically in FIG. 1. In the present embodiment, the medical imaging data processing apparatus 20 is configured to process medical imaging data. In other embodiments, the medical imaging data processing apparatus 20 may be configured to process any other appropriate data.

The medical imaging data processing apparatus 20 comprises a computing apparatus 22, which in this case is a personal computer (PC) or workstation. The computing apparatus 22 is connected to a display screen 26 or other display device, and an input device or devices 28, such as a computer keyboard and mouse.

The computing apparatus 22 is configured to obtain data sets from a data store 30. The data sets have been obtained or generated using any suitable apparatus or from any suitable source.

In some embodiments, at least some of the data can include or can be determined from medical imaging data, for instance obtained using a scanner 24. The scanner 24 may be configured to generate medical imaging data, which may comprise two-, three- or four-dimensional data in any imaging modality. For example, the scanner 24 may comprise a magnetic resonance (MR or MRI) scanner, CT (computed tomography) scanner, cone-beam CT scanner, X-ray scanner, ultrasound scanner, PET (positron emission tomography) scanner or SPECT (single photon emission computed tomography) scanner. The medical imaging data may comprise or be associated with additional conditioning data, which may for example comprise non-imaging data.

The computing apparatus 22 may receive data from one or more further data stores (not shown) instead of or in addition to data store 30. For example, the computing apparatus 22 may receive medical imaging data from one or more remote data stores (not shown) which may form part of a Picture Archiving and Communication System (PACS) or other information system.

Computing apparatus 22 provides a processing resource for automatically or semi-automatically processing the data. Computing apparatus 22 comprises a processing apparatus 32. The processing apparatus 32 comprises model training circuitry 34 configured to train one or more models; data processing circuitry 36 configured to apply trained model(s) and to perform other processes; and interface circuitry 38 configured to obtain user or other inputs and/or to output results of the data processing. Interface circuitry 38 may be further configured to generate a user interface and process user inputs when the user interacts with the user interface using the input device 28 or other input device.

In the present embodiment, the circuitries 34, 36, 38 are each implemented in computing apparatus 22 by means of a computer program having computer-readable instructions that are executable to perform the method of the embodiment. However, in other embodiments, the various circuitries may be implemented as one or more ASICs (application specific integrated circuits) or FPGAs (field programmable gate arrays).

The computing apparatus 22 also includes a hard drive and other components of a PC including RAM, ROM, a data bus, an operating system including various device drivers, and hardware devices including a graphics card. Such components are not shown in FIG. 1 for clarity.

The medical imaging data processing apparatus 20 of FIG. 1 is configured to perform methods as illustrated and/or described in the following.

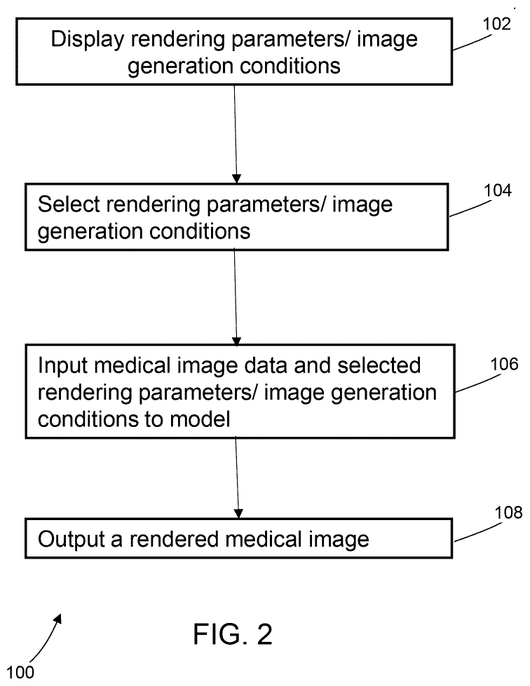

FIG. 2 is a flowchart of a method 100 according to an embodiment, for example performed using the apparatus of FIG. 1. In the method of FIG. 2, a user may interact with computing apparatus 22 to automatically or semi-automatically process medical image data.

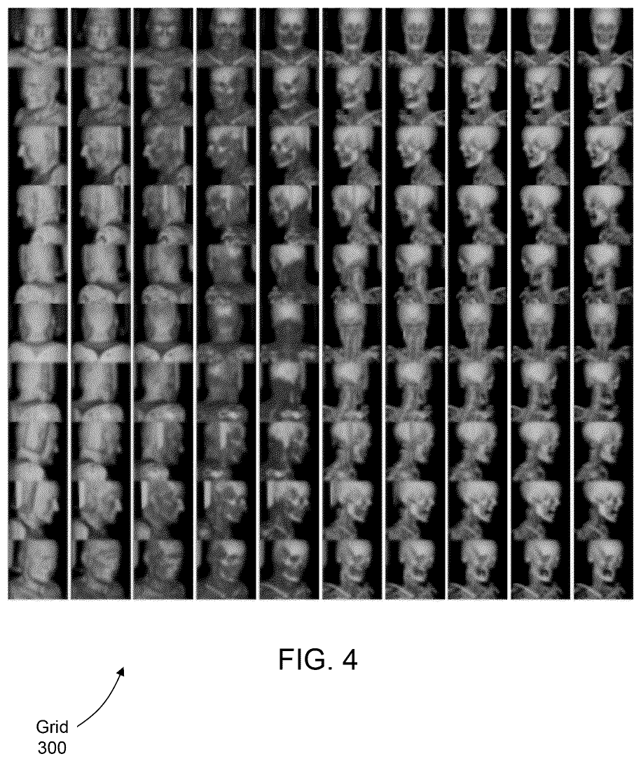

At stage 102 of the method the computing apparatus 22 displays on the display screen 26 a map representing one or more rendering parameters and/or image generation conditions. In the embodiment of FIG. 2, the map comprises a plurality of rendered images in a grid as shown in FIG. 4, discussed further below. Any other suitable map may be displayed in other embodiments, and the map is not limited to being a grid arrangement.

The images of the map in the process of FIG. 2 are rendered using a plurality of different rendering parameters/image generation conditions. The map may provide a clinically useful starting point for a user when analysing or interacting with medical image data. The map generated in the embodiment of FIG. 2 is interactive such that a user may select one or more of rendering parameters, image generating conditions, rendered image data and values associated with the rendering parameters and image generating conditions.

Further selections of parameters, conditions or images may update the contents of the map by applying the new selection of parameters or conditions to the same or new images. The map may comprise indicators which may be set by interacting with the computing apparatus using input device 28, such as a mouse or keyboard. The indicators may mark selected rendering parameters, image generation conditions and values associated with either of these. The display of the map on the display screen 26 may be initiated by the user prompting the computing apparatus to display the rendering parameters and/or image generation conditions, or a user-selected subset of these. The parameters and/or conditions displayed may comprise some or all of the rendering parameters and/or image generation conditions that may be used to process medical image data using the computing apparatus.

At stage 104, the user selects one or more rendering parameters and/or image generation conditions and provides the selected parameters/conditions to the computing apparatus 22. This may be done by setting one or more indicators associated with one or more rendering parameters and/or image generation conditions. Indicators may comprise marking a representation of a parameter/condition as ‘selected’ or interactive indicators such as sliders or rotatable ‘knobs’ to set the values associated with a parameter/condition or other suitable indication mechanisms. The user may also select a subrange of values associated with each parameter/condition using the indicators.

At stage 106, the computing apparatus 22 provides medical image data and one or more rendering parameters and/or image generation conditions to a trained machine learning model.

At stage 108, the computing apparatus 22 displays at least one rendered image and at least some medical image data such as semantic data in the form of captions, on the display screen 26. The semantic data associated with one or more images may also be displayed on the display screen 26. The captions comprising the semantic data may be overlaid on associated rendered images.

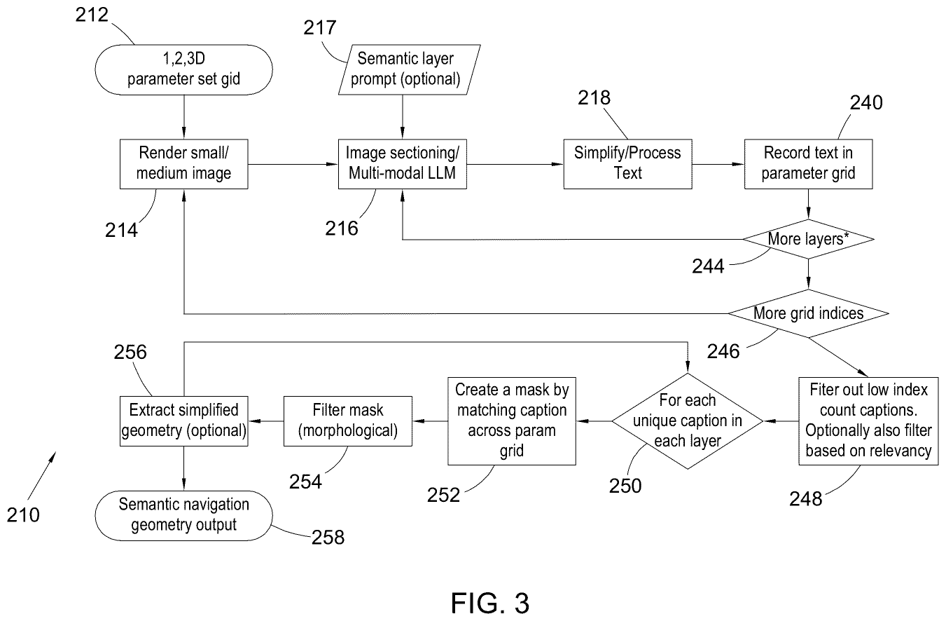

FIG. 3 is a schematic of a further method 210 of processing medical image data according to an embodiment. In step 212, medical imaging data is provided to the processing apparatus 32 (of FIG. 1). The medical imaging data may comprise three-dimensional volumetric data. The medical imaging data may comprise any imaging modality including but not limited to imaging data obtained from a magnetic resonance (MR or MRI) scanner, CT (computed tomography) scanner, cone-beam CT scanner, X-ray scanner, ultrasound scanner, PET (positron emission tomography) scanner or SPECT (single photon emission computed tomography) scanner. The imaging data may comprise two-dimensional and/or one-dimensional data. The imaging data may be in the form of a series of three, two or one-dimensional images over a period of time, such as a video format or animation. In some embodiments, the images used do not need to be high resolution images. The adequacy of low resolution images resulting in a clinically useful output visual representation for the user is an advantage of the described invention. One or more rendering parameters or other image generation conditions may also be provided to the processing apparatus 32 in step 212. One or more sets of values or a range of values associated with each rendering parameter or other image generation condition may also be provided to the apparatus.

References to image rendering parameters in described embodiments may be replaced by any suitable other image generation conditions in other embodiments.

In various embodiments, the image generation conditions may include conditions regarding segmentation, or may include a condition related to at least one of image rotation, enlargement and reduction, or viewing direction, or may include a condition related to rendering. The plurality of image generation conditions may comprise multiple types of image generation conditions.

If a range of values is provided for a rendering parameter, the processing circuitry may derive a series of values of the rendering parameter at which to render the image. The values of rendering parameters may be discrete.

In step 214, the medical imaging data is rendered based on the rendering parameters provided to or derived by the apparatus. Rendering image data may comprise processing image data to obtain further image data. Rendering may comprise filtering image data. Rendering may enhance or de-emphasise visual aspects of image data. The imaging data may be rendered using graphics processing unit (GPU) batch-rendering. The medical imaging data may be rendered for one or more values of the rendering parameters provided to the apparatus or derived by it. The medical imaging data may be rendered for each value in a set of values or a range of discrete value values of each rendering parameter for which these values are provided to or derived by the apparatus. The medical imaging data may be rendered for all provided/derived combinations of values of the rendering parameters. Each rendered image resulting from step 214 may be generated using respective different values of the rendering parameters provided to or derived by the apparatus. In this way, each medical image may be rendered for every value of every rendering parameter provided as well as every combination of value and rendering parameter provided. As an example, if ‘N’ distinct values are provided/derived for ‘M’ distinct rendering parameters used to render one original image, the total number of rendered images that result will be at least: N!/[M!(N−M)!] for example N^{M}.

In step 216, the rendered images are processed by a trained machine learning model to generate semantic data associated with the rendered image. The semantic data may describe and/or represent one or more features in the rendered image. The model may identify these features in the rendered images and assign semantic data to each identified feature. The features may comprise anatomical features and/or specific pathologies.

The trained machine learning model may be a generative Large Language Model (LLM) or other model. The trained machine learning model may perform captioning on the rendered image data. The trained machine learning model may caption the features identified in the rendered images to produce semantic data in the form of captions. The trained machine model may be trained on medical data to provide semantic data of a medical or clinical nature. The model may comprise an LLM employing a captioning vision model, for example CLIP/BLIP and such models may be used directly. The model may comprise a medically trained captioning/vision/multi-modal LLM. Alternatively or additionally, the model may comprise at least one of GPT-2, GPT-3.5, GPT-4, PaLM, LLaMa, BLOOM, Ernie, T5, Claude or Claude 2 or any suitable derivatives or developments thereof. The trained machine learning model may comprise a generative LLM which takes image data or a combination of image and text data as input and returns semantic data. The LLM may be located on a server 25 remote from the computing apparatus 22 of FIG. 1 in some embodiments. Communication between the computing apparatus 22 and the trained model may be via the internet or any other suitable communication or networking method. In such embodiments, the processing circuitry 36 may provide an application programming interface (API) that is configured to receive prompts or other input, to send the prompts or other input to the LLM or other model, and to receive responses from the LLM or other model.

In other embodiments, the trained model may be stored or implemented locally at the apparatus 22. The trained model may be implemented by the data processing circuitry 36.

The method may comprise receiving a prompt from the user in step 217. The prompt may be provided to the LLM by means of the input device 28. The prompt may modulate the processing task assigned to the LLM. The prompt may comprise at least one of text data and image data which is processed by the LLM in addition to the rendered images. The prompt may be used to guide the LLM in the processing task by providing additional context to the language model. As an example, the prompt may instruct the LLM to search for a particular anatomical feature or specific pathology. In embodiments, the prompt may instruct the LLM to search for the major anatomy in one or more images. The prompt may also modulate the format of the output of the LLM, such as the number of words and/or characters. The prompt may instruct the LLM to generate a particular number of semantic descriptions from the rendered images. The prompt may also instruct the interface circuitry 38 to generate an output display and/or user interface provided for the user during and after the completion of method 210. The prompt may also instruct the interface circuitry 38 to generate a particular user interface and define the elements of the interface to be generated.

In step 218, the semantic data obtained from the LLM may be further processed. The further processing may be performed by the LLM or by a second LLM specifically trained for further processing or by other trained model. The further processing may be performed by the processing apparatus 32 according to a predefined set of instructions. The further processing of the semantic data may comprise a simplification of the semantic output of the LLM. The simplification may comprise the reduction or redaction of text data in the semantic output of the LLM. The further processing in step 218 may be performed by a user.

In step 240, the output semantic data from step 218 is stored in a dataset. The dataset comprises the rendered images and the semantic data associated with the one or more features identified by the LLM in each rendered image. The dataset also comprises the rendering parameters used to render the medical imaging data and the values of the rendering parameters used in the rendering process. The dataset further comprises the associations between the rendering parameter values, the rendered images obtained for each value of rendering parameters, the features identified in each rendered image and the semantic data generated to identify the features. The dataset may contain information representing the presence or absence of one or more features in the rendered images, represented in the semantic data as a function of the rendering parameter values used to generate the rendered images. The dataset may be in the form of a multidimensional parameter space wherein each dimension comprises a rendering parameter used to render a medical image. Co-ordinate points along each dimension of the multidimensional parameter space may represent values of the rendering parameters used to render the image. For example, some rendered images will be rendered for discrete values of a first rendering parameter while the values for other rendering parameters will be zero. Such rendered images will be associated with co-ordinate points along the single dimension of the first rendering parameters. Other rendered images will be rendered for specific non-zero values of more than one rendering parameter simultaneously. These rendered images will be associated with co-ordinate points that do not lie on any single dimension of the multidimensional co-ordinate space.

In step 244, the semantic data collected in earlier steps is assessed for completeness. This assessment may be performed by the LLM or the second LLM or by a third LLM or other model specifically trained for assessing completeness. The assessment may be performed by the processing apparatus 32 according to a predefined set of instructions. The assessment may be performed by a user. The user may interact with the apparatus using the display screen 26 and the input device 28.

In one example, at least some of the semantic data or the dataset of step 240 may be displayed on the display screen 26 and the user may provide an input regarding the completeness of the semantic data using the input device 28. Completeness in this regard may be defined as having enough semantic information to deliver a clinically useful set of identified features to a user of the apparatus. If the semantic information is assessed to be incomplete, the method returns to step 216 and reprocesses the rendered image data as before, but with the LLM conditioned to obtain a more complete set of semantic data from the rendered images. The more complete set of semantic data may comprise a larger number of identified features and/or further descriptive detail about the identified features. If the semantic information is assessed to be complete, the method proceeds to step 246.

In step 246 the data collected in earlier steps is assessed, for example on the basis of resolution. This assessment may be performed by the LLM or the second LLM or the third LLM or by a fourth LLM or other model specifically trained for assessing resolution. The assessment may be performed by the processing apparatus 32 according to a predefined set of instructions. The assessment may be performed by a user. The user may interact with the apparatus using the display screen 26 and the input device 28. In one example, at least some of the dataset of step 240 may be displayed on the display screen 26 and the user may provide an input regarding the resolution of the semantic data using the input device 28. Resolution in this regard may be defined as the apparatus having been provided/derived enough discrete values for one or more rendering parameters to deliver a clinically useful set of identified features to a user of the apparatus. If the data is assessed to be of lower resolution that required, the method returns to step 214 and reprocesses the image data as before, but with the imaging data rendered for a larger number of discrete values of the one or more rendering parameters. The reprocessing of the image data may apply for one or more of the rendered parameters. In some examples, one or more rendering parameters may be used to re-render the images at a higher resolution whereas in other examples, all provided rendering parameters may be used to re-render the images at a higher resolution. If the resolution of the data is assessed to be adequate, the method proceeds to step 248.

In step 248, the semantic data associated with features identified in rendered images are filtered on the basis of incidence and/or relevance. Filtering on the basis of incidence may comprise filtering out semantic data with low incidence. The threshold for incidence may be set by a user. The threshold for incidence may also be updated during operation by the user. The threshold for incidence may also be adaptive and may be calculated by the processing apparatus. The adaptive threshold for incidence may be calculated in relation to the incidence of all semantic data associated with a given input set of medical image data. In this way, the processing apparatus may be able to identify dominant semantic data relating to dominant features identified in the rendered images while filtering out spurious semantic data. Similarly, the threshold for relevance may be set by a semantic understanding of the set of generated semantic data. Any of the previously described LLMs or other models may be use to ascertain the relevance of a given piece of semantic data in relation to the set of all generated semantic data. This provides an additional method for removing spurious semantic data from the generated semantic data. The output of step 248 is a dataset comprising semantic data associated with one or more features identified in each rendered image, wherein the semantic data has been further simplified (in step 218), assessed for and subsequently corrected for completeness (step 244) and resolution (step 246) and filtered based on incidence and/or relevance (step 248).

Step 250 selects an instance of semantic data associated with a feature identified in a rendered image. The semantic data may comprise a text caption generated by the LLM in step 216.

In step 252, the apparatus generates a visual representation of semantic data as a function of the rendering parameters used to generate the rendered images associated with the semantic data. The visual representation of the parameter space may comprise a plurality of dimensions, wherein each dimension represents one or more of the rendering parameters. In the current embodiment, the visual representation is in the form of regions in a parameter space that represent semantic data and wherein the regions correspond to the co-ordinate locations in the parameter space that correspond to the rendered images where the semantic data was identified by the LLM in step 216.

The representation of the semantic data in the parameter space can also be referred to as a parameter space dataset.

The regions may represent the presence or absence of the one or more features in the rendered images, represented by the semantic data. In other embodiments, other forms of visual representation may be used. The visual representation may comprise marking visually, in a multidimensional parameter space, the coordinates where one or more features were identified in the rendered images. The visual representation in other embodiments may visually mark the coordinates where one or more features were absent in the rendered images. In some embodiments, all the coordinate points wherein a particular feature was identified may be visually marked identically, such as by the use of colour or shading or other visual representation. In some embodiments, all the coordinate points that represent locations in the parameter space where a particular feature was identified may be marked such that neighbouring visual indications are joined with each other to create regions that represent the locations of the feature. Such regions may represent either the presence or the absence of a particular feature at each coordinate point in the multidimensional parameter space that the regions covers. This may be done for one or more features that comprise the dataset generated at the output of step 248. The regions may be described as masks, wherein each mask is associated with the semantic description of a feature identified in one or more rendered images. A plurality of masks that represent the same semantic description may be contiguous or non-contiguous.

In step 254, the mask or masks created in step 256 may be smoothed. The smoothing may be achieved by a filtering process. The smoothing process may comprise morphological filtering. In step 256, the filtered mask or masks may be further simplified visually. The simplification may comprise approximating the visual shape of the masks to one of several pre-defined shapes.

In step 258, the visual representation of semantic data in the multidimensional parameter space is provided to the user. The representation may be stored in memory or displayed on the display screen 26. The representation may take several different forms beyond the visual features described above. The processing circuitry may generate the visual representation of the parameter space dataset on the display screen. Regions in the representation may be annotated with the semantic data associate with them. For the case wherein only one rendering parameter is used to render the medical image data, the output may comprise a bar plot on a one-dimensional axis. The length of the bar plot may be divided into segments wherein each segment represents the regions which comprise the rendering parameter values wherein semantic data associated with a given feature is identified.

Segments may overlap where more than one feature is identified in an image at a particular ordinate point along the axis wherein the ordinate point represents the value of the one rendering parameter used to render the medical image data. For the case wherein two rendering parameters are used to render the input medical image data, the output may comprise a two-dimensional plot wherein each rendering parameter is represented as one of the dimensions of the plot. Coordinate points in such a plot represent simultaneous values of the two rendering parameters used to render the input medical image data.

Regions in the two-dimensional parameter space that represent semantic data identifying features in the rendered images may be shown in such a plot as two-dimensional masks. For a case wherein three rendering parameters are used to render the input medical image data, the output might comprise a three-dimensional plot wherein each rendering parameter is represented as one dimension of the plot. Coordinate points in such a plot represent simultaneous values of the three rendering parameters used to render the input medical image data.

Regions in the three-dimensional parameter space that represent semantic data identifying features in the rendered images may be shown as three dimensional objects or voxel masks. For the case where more than three rendering parameters are used to render the input medical image data, the visual representation may comprise reducing the dimensions and projecting the parameter space and any masks or geometries into a two or three dimensional coordinate space. The resulting two or three dimensional coordinate spaces would then have the respective properties described above.

FIG. 4 shows a two-dimensional grid 300 of rendered images. The grid may be referred to as a map, and any other suitable form of map may be used in other embodiments. In some embodiments, the grid 300 may be presented as an output to the user. In other embodiments the images are not output. The output may be presented on the display screen 26. For the embodiment of FIG. 4, the input imaging data is a three-dimensional volumetric dataset comprising voxel data. In other embodiments, such a series of images could be obtained from a two-dimensional set of images or imaging data comprising dimensions higher than three. The images comprising FIG. 4 are rendered using two rendering parameters, namely ‘rotation’ and ‘threshold’. In other embodiments, other rendering parameters may be used to render input imaging data. The images comprise images of a human head and neck. The rotation of the anatomy in this embodiment is a sagittal rotation. Sagittal rotation varies in the vertical direction in the grid illustrated in FIG. 4. It may be the case that such a rendering parameter is controlled using an input box wherein an angle of rotation may be entered, a slider which may be interacted with to change the angle of rotation or by some other interactive feature allowing a user to interact with image data. The same may be true of the second rendering parameters used to obtain the images in FIG. 4, for example threshold parameter(s). In this embodiment, threshold is related to the absorption of the illumination used for imaging by the materials of the human head and neck. A high threshold reveals the anatomy of the head and neck at a greater depth, the depth being dependent on the absorption experienced by the illumination. The threshold increases in a horizontal left-to-right direction as can be seen in the increasing depth of imaging in the left-to-right direction in FIG. 4.

FIG. 4 shows the rendered images as a function of the rendering parameters used to render them. It can be seen from FIG. 43 that the visual content of the images varies as a function of the rendering parameters used and in particular, of the values of the rendering parameters. It can further be understood from FIG. 4 that a trained LLM to which the images are provided as input, may generate a variety of semantic data to identify features in the rendered images.

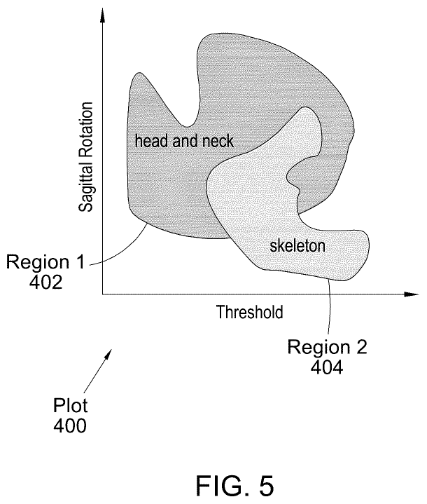

FIG. 5 represents a two-dimensional parameter space wherein rendering parameters vary along the dimensions of the plot. The parameter space illustrated in FIG. 5 may be presented as a visual output to a user. The output may be presented on the display screen 26.

In FIG. 5, sagittal rotation varies in the vertical direction while threshold varies in the horizontal direction. Two regions, Region 1 402 and Region 2 404 are disposed in the plot in FIG. 5. Each of these regions may comprise the combination of all the sets of values of rendering parameters that result in rendered images where a particular semantic description is generated by the LLM. As elaborated earlier, the semantic description generated by the LLM identifies a visual feature in the rendered image. The regions 402, 404 may hence comprise coordinate points corresponding to rendered images that when processed by the LLM, result in the LLM generating the same semantic descriptions. In other words, each coordinate point in each region 402, 404 corresponds to a rendered image which when processed by the LLM, generates a respective same semantic description identifying a feature in the rendered image. In FIG. 5, the Region 1 402 is annotated by the semantic description “head and neck”. In other embodiments, the region may not be annotated by a semantic description. In some embodiments, a legend may be made available as a visual element on the display screen 26 to identify the semantic description that corresponds to the region. In some embodiments, the legend may identify the semantic description that corresponds to the region by colour coding or shading the region in correspondence with the colour or shading represented in the legend. The annotation corresponding to Region 1 402 may represent the semantic description that the LLM generated for one or more features that were identified by the LLM in all the rendered images with coordinate positions that fall within Region 1 402. In other words, all rendered images with coordinates that fall within Region 1 402 are identified by the LLM as comprising images of the head and neck. Similarly, all rendered images with coordinate positions that fall within Region 2 404 are identified by the LLM as comprising images of a skeleton since the annotation corresponding to Region 2 404 in FIG. 5 is ‘skeleton’. Region 1 402 and Region 2 404 intersect for over a region in the parameter space illustrated in FIG. 5. This represents the simultaneous identification of a skeleton and a head and neck in the rendered images that fall within the region where Region 1 402 and Region 2 404 intersect. The parameter space of FIG. 5 is identical to the parameters space of FIG. 4 and the correspondence of the identified semantic descriptions and the rendered images may be discerned from a comparison of the two.

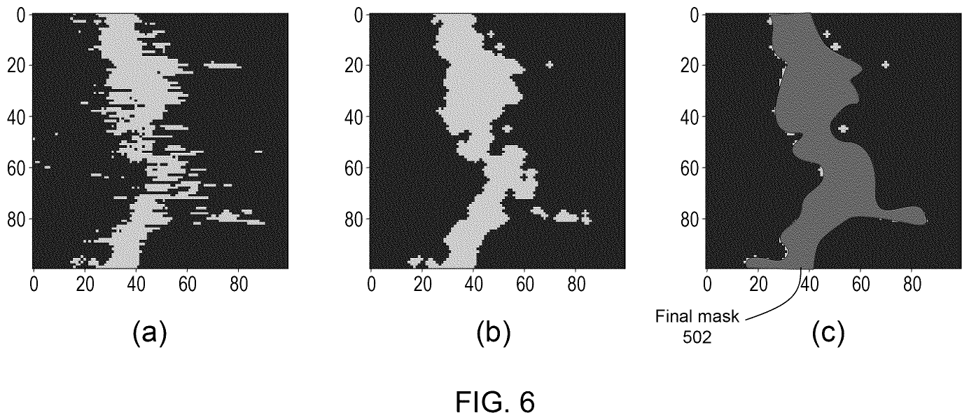

FIG. 6 shows three representations of a two-dimensional parameter space in accordance with an embodiment. FIG. 6 illustrates three embodiments of a two dimensional-parameter space which comprise visual representations of regions associated with semantic descriptions in the form of a two-dimensional plot. One or more of these figures may be presented as an output to the user. The output may be presented on the display screen 26. In each of FIG. 6 (a), 6 (b) and 6 (c), the coordinate space represents a two-dimensional parameter space wherein the dimensions of the parameter space comprise rendering parameters such as the sagittal rotation and threshold parameters of previously described examples. In FIG. 6 (a), 6 (b) and 6 (c), the axes of the plots are marked with dimensionless values which are representative of the varying values of rendering parameters along the axes. In other embodiments, the values of the rendering parameters, such as an angle in degrees or radians for sagittal rotation, may be marked on the axes. In each of these figures, regions representing the presence of a unique semantic description are illustrated as visually overlaid on the parameter space. In some embodiments, these regions may be colour coded in contrast to the background. Each figure illustrates the presence of one unique semantic description, but in other embodiments, the presence of multiple semantic descriptions may be presented in one figure.

In FIG. 6 (a) the regions illustrating a unique semantic description are shown as masks in the form of pixels in the parameter space. Each pixel represents a coordinate point in the parameter space which represents a rendered image comprising a feature identified by the LLM as having the same semantic description. Some of the pixels are shown to be non-contiguous whereas other pixels are contiguous and form larger regions. FIG. 6 (a) may be generated by starting with the dataset generated in step 248 of method 210 and creating a mask that covers all the coordinate points that represent a particular semantic description from the dataset.

The plot shown in FIG. 6 (b) may be generated by processing the plot of FIG. 6 (a). This processing may comprise morphological filtering. The morphological filtering may be an automatic process, semi-automatic process or a manual process requiring user input. The morphological filtering may be instructed to filter the image to approach the morphology of human anatomy when the medical imaging data is that of human anatomy. The processing may comprise filtering or smoothing of the plot of FIG. 6 (a) to generate a mask. It can be seen that the regions illustrating the unique semantic description in FIG. 6 (b) are more contiguous and with smoother boundaries than the equivalent regions in FIG. 6 (a). In other embodiments, or in other instances of automatic filtering, the generated shapes may be more or less contiguous and the boundaries of the regions may be more or less smooth.

The plot shown in FIG. 6 (c) may be generated by processing the plot of FIG. 6 (b) with the aim of obtaining a simplified final mask 502 depicting regions that represent a particular semantic concept. This processing may comprise further smoothing and filtering. The processing may include the abandonment of one or more non-contiguous regions from FIG. 6 (b) from the final mask. The processing may also include coordinate points where the semantic description is not present into the final mask. It can be seen that the final mask in FIG. 6 (c) is overlaid on the mask generated in FIG. 6 (b). The boundaries of the final mask may be smoother than the boundaries of the mask of FIG. 6 (b) as can be seen in FIG. 6 (c) for this particular embodiment. Some regions that were included in the mask of FIG. 6 (b) are not included in the final mask, while some regions that were not included in the mask of FIG. 6 (b) are now included in the final mask. In other embodiments, the mask may have more or less smooth edges, include more or less regions not included in the mask of FIG. 6 (b) and exclude more or less regions included in the mask of FIG. 6 (b). While the final mask of FIG. 6 (c) is contained within a single boundary, in other embodiments, the mask may comprise two or more non-contiguous regions with non-overlapping boundaries.

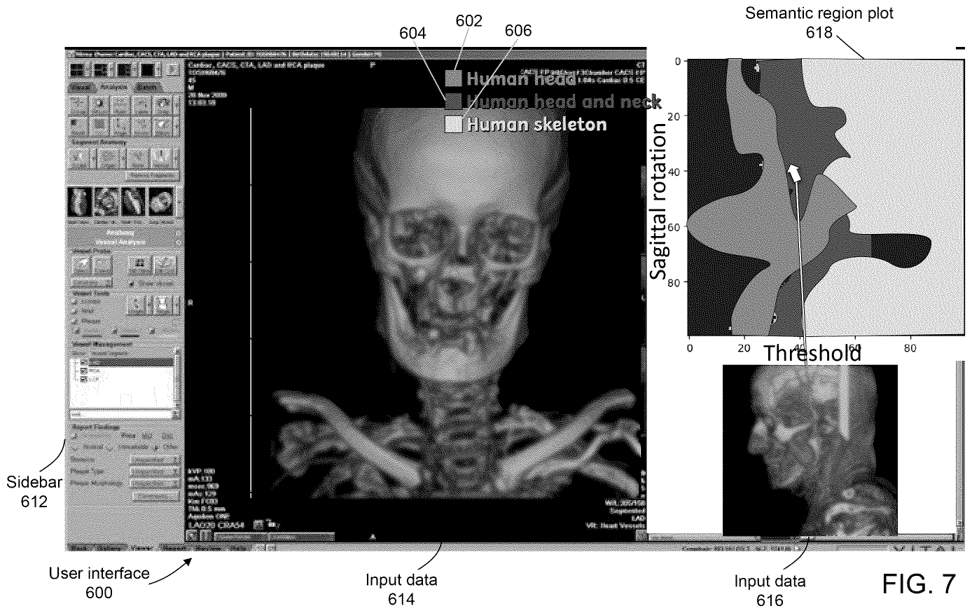

FIG. 7 is an image of a user interface in accordance with an embodiment. FIG. 7 shows an image of a user interface 600 that may be used to present one or more visual results to the user on the display screen 26 or other display device and receive input from the user using the input device 28 or other input device. The input from the user may modulate the contents of the display screen 26 or other display device.

The embodiment of FIG. 7 includes main elements (612, 614, 616, 618), for example in the form of four windows or other areas, and a legend (602, 604, 606). Other embodiments may comprise more or fewer elements as well as elements not shown in FIG. 7. A sidebar 612 shown in FIG. 7 may function as an input element and/or an output element of the user interface. The sidebar may illustrate detailed information about the remaining elements of the user interface. The sidebar may also be used to select the remaining elements on the display screen as well as to alter the composition of elements on the screen. The sidebar may be used to select semantic information to display on the screen as well as to select what subset of the dataset to display. Input data 614 and 616 shows the input medical imaging data for the particular embodiment of FIG. 7.

In FIG. 7, the input data is a three dimensional image but in other embodiments, other modalities of imaging data may be displayed. The input data may be navigable by user interaction, such as being rotatable by using a click and drag movement on a mouse or by using directional buttons on a keyboard. The input data may be processed before being displayed on the display screen, for example, the input data may be rendered before it is displayed on the screen. Input data 614 shows rendered image wherein the image is at least rendered to a high value of threshold while input data 616 shows a rendered image wherein the image is at least rendered to a low value of threshold and is rotated with respect to the image in input data 614. Semantic region plot 618 shows a two dimensional parameter space wherein the axes represent varying values of sagittal rotation and threshold rendering parameters. In plot 618, the vertical axis represents variation in sagittal rotation while the horizontal axis represents variation in value of the ‘threshold’ rendering parameter. Regions representing three unique semantic descriptions are disposed in the parameter space in layers. A legend comprising semantic captions (602, 604, 606) representing features identified by the LLM in the input data is overlaid in the display. The semantic plot 618 may be interactive such that user selection of one or more semantic captions may bring them forward in the layered configuration or may toggle their visibility.

An arrow is included in the semantic plot 618 of FIG. 7. The arrow may be positioned at any point on the screen. The arrow is used to select an area on the semantic plot 618. The semantic plot 618 of FIG. 7 comprises three regions which may comprise or correspond to masks as shown in FIG. 6 and represent semantic descriptions associated with features obtained from the medical image data. In FIG. 7, the arrow 620 is co-located with a regions associated with the semantic concept “Human head and neck” based on the legend entry 604. Co-location of the arrow 620 with an area labelled according to a semantic concept may result in the computing apparatus 22 displaying only the rendered images (and optionally the corresponding semantic data) from the processed data that are also associated with the respective semantic concept. The non-rendered input images associated with the semantic concept may also be displayed. This may allow the user to access subsets of rendered or non-rendered images based on the semantic concepts associated with them and selected visually by the arrow 620 on semantic plot 618. For example, moving the arrow 620 to the a different region in semantic plot 618 may cause the computing apparatus to display images associated with “human head” and moving the arrow to a further region may display images associate with “human skeleton”.

One or more images from the set of input images and/or the rendered images may also be displayed in the user interface. Any of the illustrations of FIGS. 4, 5 and 6 may be included as elements of the user interface display. Either or both of input data 614 and input data 616 may be used to show thumbnails of corresponding input rendered images when, for example, a mouse pointer is made to hover over one or more coordinate points in the parameter space.

Although embodiments have been described in which threshold/level has been used for a horizontal axis and the sagittal rotation has been used for a vertical axis in the map or representation, any other desired image generation parameters can be used for the axes, or otherwise represented, in the map or other representation in other embodiments. For example, segmentation parameters (e.g. presence or absence of a particular anatomical feature or parameter) or one or more of image rotation, enlargement and reduction, or viewing direction, could be used as axes for the map or other representation.

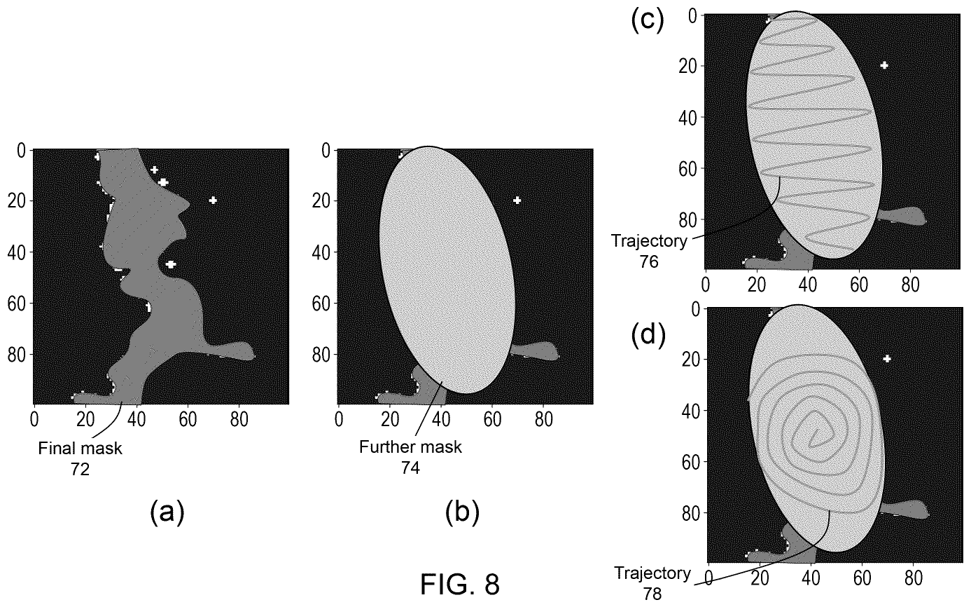

FIG. 8 (a) to 8 (d) show four representations of a parameter space in accordance with an embodiment. FIG. 8 (a) to 8 (d) shows four plots of two-dimensional parameter spaces. The output may be presented on the display screen 26. In each of FIG. 8 (a) to 8 (d), the coordinate space represents a two-dimensional parameter space wherein the dimensions of the parameter space comprise rendering parameters such as the sagittal rotation and threshold parameters of previously described examples. In FIG. 8 (a) to 8 (d), the axes of the plots are marked with dimensionless values which are representative of the varying values of rendering parameters along the axes. In other embodiments, the values of the rendering parameters, such as an angle in degrees or radians for sagittal rotation, may be marked on the axes. In each of these figures, regions representing the presence of a unique semantic description are illustrated as visually overlaid on the parameter space. In some embodiments, these regions may be color coded in contrast to the background. Each figure illustrates the presence of one unique semantic description, but in other embodiments, the presence of multiple semantic descriptions may be presented in one figure.

FIG. 8 (a) shows a mask, such as the mask generated in FIG. 6 (c) with smooth boundaries disposed in the parameter space.

FIG. 8 (b) shows the mask of FIG. 8 (a) with a further mask overlaid. The further mask is a simpler shape than the mask of FIG. 8 (a), for example, because it has smoother edges and because it is symmetric. The further mask is depicted as one of oval shape in FIG. 8 (b). There is, however, no restriction on the shape and it can be a shape of greater complexity that the mask in the lower layer (the mask of FIG. 8 (a)). It is however preferable that the further mask covers, in terms of area, some or most of the mask in the lower layer and that the mask is suitable for the functions described below in relation to FIG. 8(c) and 8 (d).

FIG. 8 (c) and 8 (d) show trajectories contained within the further mark of FIG. 8 (b). While one trajectory is shown to have a zig zag shape (FIG. 8 (c)) and the other is in a substantially spiral shape (FIG. 8 (d)), there is no restriction on the configuration of the trajectories. It is preferable however, that the trajectory is smooth and traverses a substantial portion of the further mask and that it is substantially spread over the area of the further mask. The trajectory can be used to animate a series of rendered images that are associated with coordinate points that coincide with the trajectory as it traverses the parameter space. The series of images may follow the sequence of coordinate points that coincide, or substantially coincide with the coordinate points that comprise the trajectory. The series of image may follow some other sequence while being comprised of rendered images associated with coordinate points that coincide, or substantially coincide with the coordinate points that comprise the trajectory. The trajectory and the series of images comprising the animation may be generated automatically by the processing apparatus or involve user input. In this way, the user may choose a particular semantic caption and be presented an animation comprising rendered images that show features of anatomy described by the semantic caption exclusively, or at least substantially. Since there is no restriction on the shape and extent of the trajectory, the user may also use the processing apparatus to define a trajectory in a parameter space where rendering parameters change in a predictable way while viewing features identified by a semantic caption of interest. As an example, the user may prompt the processing circuitry to generate an animation comprising rendered images substantially of the human head with varying values of threshold while the sagittal rotation is kept constant or within a specified range.

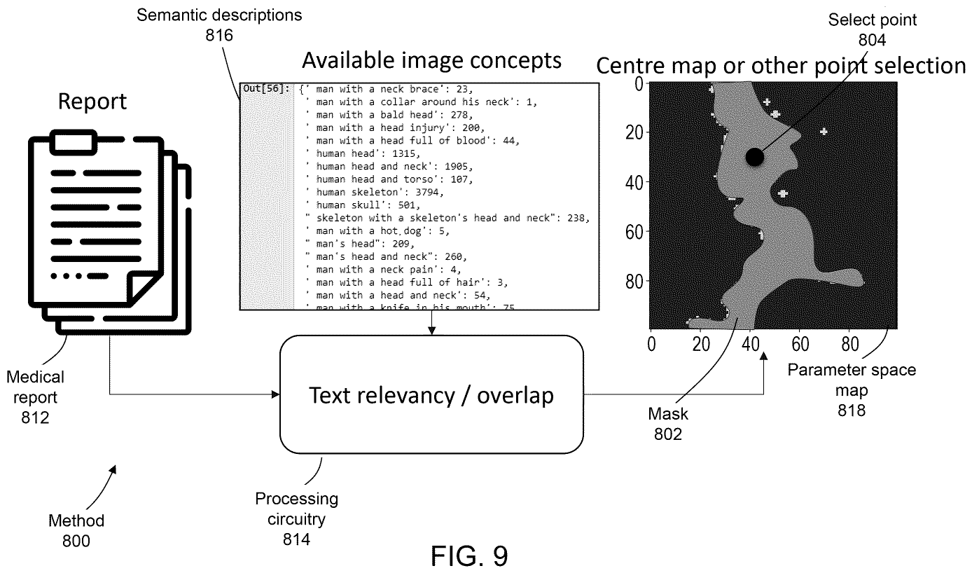

FIG. 9 is a schematic of a method in accordance with an embodiment. FIG. 9 illustrates a method 800 for generating a clinically useful visual representation of rendered image data for a user. Method 800 may be used as an extension of method 210 and relies on data generated during method 210. Method 800 may also be a standalone method exclusive of method 210.

In method 800, a medical report 812 is provided to the processing apparatus. Medical report 812 may be a clinical report and may contain semantic data and/or image data. Medical report 812 is provided to a processing circuitry 814. Method 800 may use its own processing circuitry or share processing circuitry with the processing apparatus 32 of FIG. 1. Processing circuitry 814 may comprise an LLM trained to generate semantic data from semantic and/or image data inputs. The LLM may be the LLM used in step 216 of method 210 or may be an alternate LLM. Processing circuitry 814 also receives a data comprising semantic descriptions 816 as an input. The semantic descriptions 816 may be obtained using method 210. The semantic descriptions 816 provided to processing circuitry 814 may be directly generated from the LLM in method 210. The semantic

descriptions 816 may be filtered as in step 248 of method 210 before being provided to the processing circuitry 814. Processing circuitry 814 is configured to find a semantic overlap between the contents of the medical report and the semantic descriptions provided to it. In some embodiments, judgement of semantic overlap comprises matching semantic data between the semantic descriptions 816 and the medical report 812. The medical report may be processed by the processing circuitry 814 to obtain a set semantic descriptions for the text and/or image data in the medical report. The output of the processing circuitry 814 comprises a set of semantic data that is the subset of the semantic descriptions 816 which is relevant to or matches semantic data contained in the medical report 812. Method 800 can hence be seen as filtering semantic descriptions obtained using method 210 on the basis of relevance to the medical report.

FIG. 9 further includes a representation of a two-dimensional parameter space. This parameter space may be the same as or similar to the parameter spaces of any of FIGS. 5-8 or derived using the same or similar methods. The parameter space may be two dimensional and comprise a mask 802 which may be derived using methods described earlier. The set of semantic data, filtered on the basis of relevance to the medical report 812 is used by the processing circuitry 814 to select or visually mark a portion of the parameter space in parameter space map 818. In FIG. 9, this is shown by selected point 804. Any other suitable indicator may be used for the selected point 804 in alternative embodiments. The setting of the indicator for example on the map can be used to select one or more of the image rendering parameters or other image generation conditions in any suitable manner.

The selected point 804 is disposed at a coordinate point in the parameter space that corresponds to one the semantic data filtered based on relevance to the medical report 812. While only one selected point 804 is shown in FIG. 9, other embodiments may generate more than one selected point, possibly due to a higher number of matches between the semantic information contained in the medical report 812 and the semantic descriptions 816. The one or more selected points may be used to automate views for a user. The processing apparatus may generate a sequence of images that cycle through the images that correspond to the coordinate locations of the selected points. The processing apparatus may generate an image to display to the user wherein the selected points are marked or a sequence of images where that section of the parameter space is magnified. The one or more selected points may also be used as coordinate points that a trajectory (such as those described in reference to FIG. 8) passed through in order to automatically animate semantic concepts considered relevant on the basis of the medical report.

According to certain embodiments there is provided a medical visualisation apparatus or method comprising an image captioning model or multi-modal LLM, a one or multi-dimensional rendering parameter set sampled to create grids images representing the varying parameters, in which the image grid samples are fed through the image captioning, simplified and plotted as semantic concepts back in the original grid. Each relevant unique image concept may then be converted into a mask and optionally turned into sets of geometric pattern mapping each concepts into a parameter region.

The multi-mask plot or geometric plot may be provided to the user as a user interface (UI) element. Clicking on the plot may set the parameter combination. The plot may comprise, for example, a 1D plot, for instance a bar plot of semantic concepts on a 1D axis. The plot may comprise, for example, a 2D plot, for instance an image-based plot where semantic concepts are represented as 2D geometry or a 3D mask. The plot may comprise, for example, a 3D plot, for instance a navigable 3D scene where semantic concepts are represented by 3D geometry or voxel masks. The semantic concepts may exist as N (e.g. N>3) dimensional objects and the space would be an active dimensionality reduction view projecting the N dimensional geometry into 2D/3D.

Multiple prompts and/or instructions may be provided to the captioning model. The output would then extend to multiple layers of geometry shown to the user as a multi-layered composited view. The corresponding image may be shown as a thumbnail when hovering over the parameter geometry plot. The text semantic concepts may be plotted into the geometric regions they represent. The text to region mapping may be provided as a separate legend. The mask may be filtered before converting into geometry in order to create a more consistent space. For instance, morphological filters that comprise an opening operation followed by a closing operation may be used.

The fitted geometry may be simplified in order to reduce the visual complexity of the plot. The geometry may be further simplified and trajectories may be plotted within a semantic concepts space to create automated animations. The animations may continue between concepts and starts by creating parameter paths that smoothly connect on a semantic concept boundary. Parameter plot topics may be selected based on relevancy in regard to a text section/report. A central point to the selected semantic concept would then serve as the basis for an automatically generated image to be shown or attached to the text section/report. A parameter trajectory may be used with the automatically selected semantic concept geometry in order to create an automatic animation.

According to certain embodiments there is provided a method comprising, or a medical image processing apparatus comprising processing circuitry configured to:
receive medical image data;
receive one or more image rendering parameters;
process the medical image data to obtain a set of rendered images, wherein each image of the set is generated using respective different values of the one or more rendering parameters;
for each image of the set, process the rendered image using a trained machine learning model to obtain semantic data representing one or more features in the rendered image; and
generate a parameter space dataset that represents the presence or absence of the one or more features in the rendered images as a function of the rendering parameter values used to generate the rendered images.

Whilst particular circuitries have been described herein, in alternative embodiments functionality of one or more of these circuitries can be provided by a single processing resource or other component, or functionality provided by a single circuitry can be provided by two or more processing resources or other components in combination. Reference to a single circuitry encompasses multiple components providing the functionality of that circuitry, whether or not such components are remote from one another, and reference to multiple circuitries encompasses a single component providing the functionality of those circuitries.

Whilst certain embodiments are described, these embodiments have been presented by way of example only, and are not intended to limit the scope of the invention. Indeed, the novel methods and systems described herein may be embodied in a variety of other forms. Furthermore, various omissions, substitutions and changes in the form of the methods and systems described herein may be made without departing from the spirit of the invention. The accompanying claims and their equivalents are intended to cover such forms and modifications as would fall within the scope of the invention.

1. A medical image data processing method comprising:
displaying a map that represents a plurality of image rendering parameters or other image generation conditions;
setting an indicator to select one or more of the image rendering parameters or other image generation conditions;
inputting medical image data and the selected one or more of image rendering parameters or other image generation conditions into a model; and
outputting a rendered medical image or other medical image data generated using the selected one or more of image rendering parameters or other image generation conditions.

2. The method of claim 1, wherein at least one of:
a) the model comprises at least one of an image captioning model and multi-modal LLM;
b) the model is trained using a plurality of image generation conditions and a plurality of medical image data generated under the plurality of image generation conditions;
c) the map includes anatomical information;
d) the plurality of image generation conditions include conditions regarding segmentation;
e) the plurality of image generation conditions include a condition related to at least one of image rotation, enlargement and reduction, and viewing direction;
f) the plurality of image generation conditions include a condition related to rendering; or
g) the plurality of image generation conditions comprise multiple types of image generation conditions.

3. The method of claim 1, wherein
the map comprises a set of rendered images, wherein each image of the set is generated using respective different values of the one or more rendering parameters, and the method comprises:
for each image of the set, processing the rendered image using the model or a further model to obtain semantic data representing one or more features in the rendered image; and
generating a parameter space dataset that represents the presence or absence of the one or more features in the rendered images as a function of the rendering parameter values used to generate the rendered images.

4. The method of claim 3, comprising providing an output comprising a visual representation of the parameter space dataset.

5. The method of claim 3, wherein the visual representation of the parameter space comprises a plurality of dimensions, each dimension representing one or more of the rendering parameters.

6. The method of claim 3, wherein the visual representation of the parameter space comprises regions which represent the presence or absence of the one or more features in the rendered images.

7. The method of claim 6, wherein at least one of the regions is subject to one or more of a smoothing or other morphological process.

8. The method of claim 3, comprising displaying, upon selection of at least one feature by a user, at least one rendered image corresponding to at least one point in the parameter space wherein the at least one selected feature is present.

9. The method of claim 8, wherein the at least one rendered image comprises a series of rendered images corresponding to a series of points in the parameter space, forming a trajectory in the parameter space.

10. The method of claim 9, comprising displaying the images comprising the series of rendered images in a sequence which corresponds to the sequence of points comprising the trajectory through the parameter space.

11. The method of claim 3, comprising filter semantic data based on one or more of incidence rate, relevance and other criteria.

12. The method of claim 11 wherein relevance of semantic data is determined based on at least one further provided image and/or document.

13. The method of claim 3, comprising providing a user interface configured such that the selection of one or more rendering parameters by the user causes display of a corresponding view of the parameter space.

14. The method of claim 13 wherein the selection of one or more rendering parameters comprises a selection of a set of values of one or more of the rendering parameter.

15. The method of claim 13, wherein the selection of one of more parameters is performed by the user interacting with a displayed view of the parameter space.

16. The method of claim 3, wherein the feature represented in the semantic data is one or more of an anatomical feature, a pathological feature or other feature.

17. The method of claim 3, wherein the model or the further model comprises at least one of a multimodal language model, a large language model (LLM) employing a captioning vision model, GPT-2, GPT-3.5, GPT-4, PaLM, LLaMa, BLOOM, Ernie, T5, Claude or Claude 2 or any suitable derivatives or developments thereof.

18. A medical image data processing apparatus comprising processing circuitry configured to:
display a map that represents a plurality of image rendering parameters or other image generation conditions;
set an indicator to select one or more of the image rendering parameters or other image generation conditions;
input medical image data and the selected one or more of image rendering parameters or other image generation conditions into a model; and
output a rendered medical image or other medical image data generated using the selected one or more of image rendering parameters or other image generation conditions.

19. An medical image data processing apparatus according to claim 18, wherein model is stored at a remote server or in the cloud, and the inputting of the medical image data and the selected one or more of image rendering parameters or other image generation conditions comprises sending the medical image data and the selected one or more of image rendering parameters or other image generation conditions to the remote server or the cloud.

20. A non-transitory computer-readable medium storing computer-readable instructions that are executable to:
display a map that represents a plurality of image rendering parameters or other image generation conditions;
set an indicator to select one or more of the image rendering parameters or other image generation conditions;
input medical image data and the selected one or more of image rendering parameters or other image generation conditions into a model; and
output a rendered medical image or other medical image data generated using the selected one or more of image rendering parameters or other image generation conditions.
